(19) United States
(12) Patent Application Publication
(10) Pub. No.: US 2026/0224128 A1
(43) Pub. Date: Aug. 6, 2026

(54) NON CONTACT VITAL SIGN DATA MONITORING METHOD BASED ON MILLIMETER WAVE RADAR

(71) Applicant: Tinglan Technology (Shenzhen) Co., Ltd., Shenzhen (CN)

(72) Inventors: Donghong HUANG, Shenzhen (CN); Yuan DONG, Shenzhen (CN)

(73) Assignee: Tinglan Technology (Shenzhen) Co., Ltd., Shenzhen (CN)

(21) Appl. No.: 19/564,149

(22) Filed: Mar. 11, 2026

Related U.S. Application Data
(63) Continuation of application No. PCT/CN2024/072532, filed on Jan. 16, 2024.

(30) Foreign Application Priority Data
Sep. 12, 2023 (CN) 2023111764569

(51) Int. Cl. A61B 5/05; A61B 5/00; A61B 5/0205; A61B 5/024; A61B 5/08

(52) U.S. Cl. CPC A61B 5/05; A61B 5/0205; A61B 5/0816; A61B 5/7221; A61B 5/7225; A61B 5/7257; A61B 5/726; A61B 5/02444

(57) ABSTRACT

The present invention discloses a non-contact vital sign data monitoring method, system, and computer-readable storage medium based on millimeter wave radar. The method comprises: receiving echo signals in a radar detection space, and selecting static test subjects as target objects from the radar detection space based on the echo signals; Calculate the spatial position information of the target object within the radar detection space based on its echo signal; Determine the number of target objects equidistant from the radar based on spatial location information. The non-contact vital sign data monitoring method based on millimeter wave radar of the present invention has the advantages of non-contact monitoring, strong monitoring reliability, and low cost.

### CROSS-REFERENCE TO RELATED APPLICATIONS

The application claims priority to Chinese patent application No. 2023111764569, filed on Sep. 12, 2023, the entire contents of which are incorporated herein by reference.

### TECHNICAL FIELD

The present invention relates to the field of non-contact vital sign data monitoring technology, particularly to a non-contact vital sign data monitoring method, system, and computer-readable storage medium based on millimeter wave radar.

### BACKGROUND

With the continuous advancement of technology, the demand for contactless vital sign monitoring technology is constantly increasing. This technology can be used in multiple fields such as healthcare, health monitoring, emergency rescue, and safety applications. Traditional vital sign monitoring methods often require contact with patients or target objects, which may be inconvenient and uncomfortable, especially in special environments.

The non-contact vital sign monitoring method based on radar technology has attracted widespread attention. Radar systems can detect vital signs such as movement and breathing of target objects without direct contact with them by sending and receiving radio wave signals. This method helps to reduce the invasiveness of the target object and improve the convenience and comfort of monitoring.

However, although radar technology has made significant progress in the field of contactless vital sign monitoring, in some cases, such as when there are multiple people in the radar detection space, there may be multiple situations at the same distance, resulting in overlapping respiratory signals and heart rate signals that cannot be accurately distinguished. Therefore, the reliability of radar in multi person respiratory and heart rate detection is poor.

### SUMMARY

The present embodiment of the application aims to improve the reliability of vital sign monitoring for multiple target objects by providing a non-contact vital sign data monitoring method based on millimeter wave radar.

To achieve the above objectives, the embodiments of the present application provide a non-contact vital sign data monitoring method based on millimeter wave radar, comprising:
Receive echo signals within the radar detection space and select static test subjects as target objects from the radar detection space based on the echo signals;
Calculate the spatial position information of the target object in the radar detection space based on its echo signal, which includes the distance information, azimuth information, and elevation information of the target unit compared to the radar;
Determine the number of target objects equidistant from the radar based on the spatial location information provided;
If there are at least two target objects equidistant from the radar in the detection space, calculate the phase information based on the azimuth and elevation information of the target objects;
Calculate the vital sign data of the target object based on the phase information, wherein the vital sign data includes at least one of respiratory rate and heart rate.

In one embodiment, the millimeter wave radar has multiple virtual antennas;

Selecting static personnel as target objects from the radar detection space based on the echo signals, including:
Perform distance dimensional fast Fourier transform and Doppler dimensional fast Fourier transform on the echo signal to obtain distance Doppler data for each object in the thunderstorm detection space;
Perform incoherent accumulation on the range Doppler data of all virtual antennas of the radar to obtain the accumulated range Doppler data;
Perform two-dimensional constant false alarm detection on the accumulated distance Doppler data to extract objects with Doppler 0 from the radar detection space as candidate objects;
Select data with Doppler 0 from any virtual antenna and perform multi frame data accumulation processing to obtain slow Doppler dimensional distance Doppler data;
Perform static filtering on the distance Doppler data of the slow Doppler dimension to remove completely stationary background objects from the candidate objects, and use the remaining candidate objects as the target objects.

In one embodiment, calculating the spatial position information of the target object in the radar detection space based on the echo signal of the target object includes:
Perform distance constant false alarm detection on the slow Doppler distance Doppler data of the target object, and calculate the distance unit of the target object compared to the radar;
Extract all antenna data of the target object from the distance Doppler data in the fast Doppler dimension;
Based on all antenna data of the target object, use the azimuth elevation joint angle measurement algorithm to calculate the azimuth and elevation angles of the target object.

In one embodiment, the azimuth information includes azimuth frequency domain data, and the elevation information includes elevation frequency domain data;

Extracting phase information based on the azimuth and elevation information of the target object, including:
Extract phase information at the peak values of the azimuth frequency domain data and the elevation frequency domain data, respectively.

In one embodiment, before calculating the vital sign data of the target object based on the phase information, if there is only one target object in the radar detection space, or if there are multiple target objects that are not equidistant from the radar, the method further comprises:
Calculate phase information based on the distance information of the target object.

In one embodiment, the distance information includes Doppler distance data;

Calculate phase information based on the distance information of the target object, including:
Calculate phase information based on the distance unit data corresponding to Doppler 0 in the Doppler distance data.

In one embodiment, calculating the respiratory rate of the target object based on the phase information comprises:
Accumulate multiple frames of data on the phase information to obtain phase change data of the target object;
Using a 6th order Butterworth filter designed based on the frequency range of respiratory rate to filter the phase change data of the target object and extract respiratory waveform data;
Perform fast Fourier transform on the respiratory waveform data to extract the respiratory cycle and calculate the respiratory frequency of the target object.

In one embodiment, calculating the heart rate of the target object based on the phase information comprises:
Accumulate multiple frames of data on the phase information to obtain phase change data of the target object;
Using a 6th order Butterworth filter designed based on the frequency range of heart rate to filter the phase change data of the target object and extract heart rate waveform data;
Perform wavelet transform on the heart rate waveform data and then perform fast Fourier transform to extract the heart rate cycle, in order to calculate the heart rate of the target object.

To achieve the above objectives, the embodiments of the present application also propose a non-contact vital sign data monitoring system based on millimeter wave radar, comprising a memory, a processor, and a non-contact vital sign data monitoring program based on millimeter wave radar stored on the memory and executable on the processor. When the processor executes the non-contact vital sign data monitoring program based on millimeter wave radar, it implements any of the non-contact vital sign data monitoring methods based on millimeter wave radar as described above.

To achieve the above objectives, the present embodiment also proposes a computer-readable storage medium, which stores a non-contact vital sign data monitoring program based on millimeter wave radar. When the non-contact vital sign data monitoring program based on millimeter wave radar is executed by a processor, it implements the non-contact vital sign data monitoring method based on millimeter wave radar as described in any one of the above.

The non-contact vital sign data monitoring method based on millimeter wave radar in the technical solution of this application introduces azimuth and elevation information to obtain phase information when multiple target objects equidistant from the radar are detected in space. Based on the obtained phase information, the respiratory frequency and/or heart rate of each target object are calculated to achieve non-contact vital sign data monitoring. Therefore, compared with traditional contact vital sign monitoring methods or millimeter wave radar vital sign monitoring methods, the monitoring method of this application can not only achieve non-contact monitoring, but also effectively distinguish and monitor the respiratory frequency and/or heart rate of multiple equidistant target objects, improving the monitoring of vital sign data for multiple target objects. The reliability. Even so, the monitoring method of the present technical solution has advantages such as non-contact monitoring and strong monitoring reliability.

### BRIEF DESCRIPTION OF DRAWINGS

In order to provide a clearer explanation of the embodiments of the present invention or the technical solutions in the prior art, a brief introduction will be given to the accompanying drawings required for the description of the embodiments or the prior art. It is obvious that the accompanying drawings described below are only some embodiments of the present invention. For those skilled in the art, other drawings can be obtained based on the structures shown in these drawings without creative labor.

FIG. 1 is a module structure diagram of an embodiment of a non-contact vital sign data monitoring system based on millimeter wave radar according to the present invention;

FIG. 2 is a schematic diagram of the antenna array layout of a millimeter wave radar in an embodiment of a non-contact vital sign data monitoring system based on millimeter wave radar according to the present invention;

FIG. 3 is a flowchart of an embodiment of a non-contact vital sign data monitoring method based on millimeter wave radar according to the present invention;

FIG. 4 is a schematic diagram of the module for obtaining RD maps through incoherent accumulation of 12 antennas of the millimeter wave radar in the first embodiment of the non-contact vital sign data monitoring method based on millimeter wave radar according to the present invention;

FIG. 5 is a schematic diagram of the module for 2D CFAR detection on RD map in the first embodiment of the non-contact vital sign data monitoring method based on millimeter wave radar of the present invention;

FIG. 6 is a schematic diagram of the module for accumulating and processing multiple frames of data from RD maps to obtain slow Doppler dimensional RD maps in the first embodiment of the non-contact vital sign data monitoring method based on millimeter wave radar according to the present invention;

FIG. 7 is a schematic diagram of the module for calculating the joint azimuth elevation angle of all antenna data of the target object in the first embodiment of the non-contact vital sign data monitoring method based on millimeter wave radar according to the present invention;

FIG. 8 is a flowchart of another embodiment of a non-contact vital sign data monitoring method based on millimeter wave radar according to the present invention.

The implementation, functional characteristics, and advantages of the present invention will be further explained with reference to the accompanying drawings in conjunction with the embodiments.

The objective implementation, functional features and advantages of the present disclosure are further illustrated with reference to the accompanying drawings by using the embodiments.

### DETAILED DESCRIPTION OF THE EMBODIMENTS

It should be understood that the specific embodiments described herein are only used to explain the present invention and are not intended to limit the present invention.

In order to better understand the above technical solution, the exemplary embodiments of the present disclosure will be described in more detail below with reference to the accompanying drawings. Although exemplary embodiments of the present disclosure are shown in the accompanying drawings, it should be understood that the present disclosure may be implemented in various forms and should not be limited by the embodiments described herein. On the contrary, these embodiments are provided to enable a more thorough understanding of this disclosure and to fully convey the scope of this disclosure to those skilled in the art.

It should be noted that any reference symbols located between parentheses should not be constructed as limitations on the claims. The use of “including” in the text does not exclude the existence of components or steps that are not listed in the claims. The quantity word ‘one’ or ‘one’ before the component does not exclude the existence of multiple such components. The present invention can be implemented by means of hardware comprising several different components and by means of a properly programmed computer. In the unit claims listing several devices, several of these devices may be specifically embodied through the same hardware item. The use of words such as ‘first’, ‘second’, and ‘third’ does not indicate any order and can be interpreted as names.

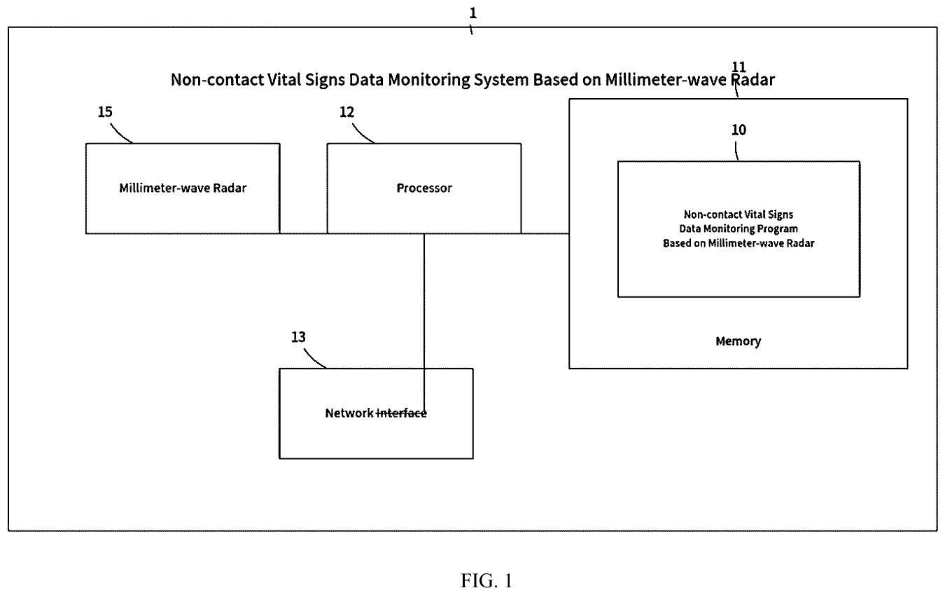

As shown in FIG. 1, FIG. 1 is a schematic diagram of the structure of a non-contact vital sign data monitoring system 1 based on millimeter wave radar according to an embodiment of the present invention. The monitoring system of the present invention is specifically designed to detect vital sign data of stationary targets

As shown in FIG. 1, the monitoring system 1 includes: millimeter wave radar 15, memory 11, processor 12, and network interface 13.

In this embodiment, the millimeter wave radar 15 adopts a 3Tx4Rx RF chip and includes 3 transmitting antennas and 4 receiving antennas. This antenna configuration allows the radar system to simultaneously transmit and receive multiple signals, thereby helping to improve the quality and reliability of the signals.

Furthermore, the millimeter wave radar adopts the TD-MIMO signal transmission mode, with three transmitting antennas transmitting signals in a time-division multiplexing manner and four receiving antennas simultaneously receiving signals.

Instant Division Multiple Access Multiple Input Multiple Output (TD-MIMO) refers to the signal transmission mode adopted by millimeter wave radar, which allows three transmitting antennas to transmit signals in a time-division multiplexing manner. This means that they take turns sending signals at different time intervals to avoid signal interference and improve system capacity and efficiency.

The four receiving antennas of millimeter wave radar simultaneously receive signals, which helps to improve the sensitivity and robustness of signal reception. Simultaneously receiving also allows the system to better distinguish and track vital sign signals of different target objects in a multi-target environment.

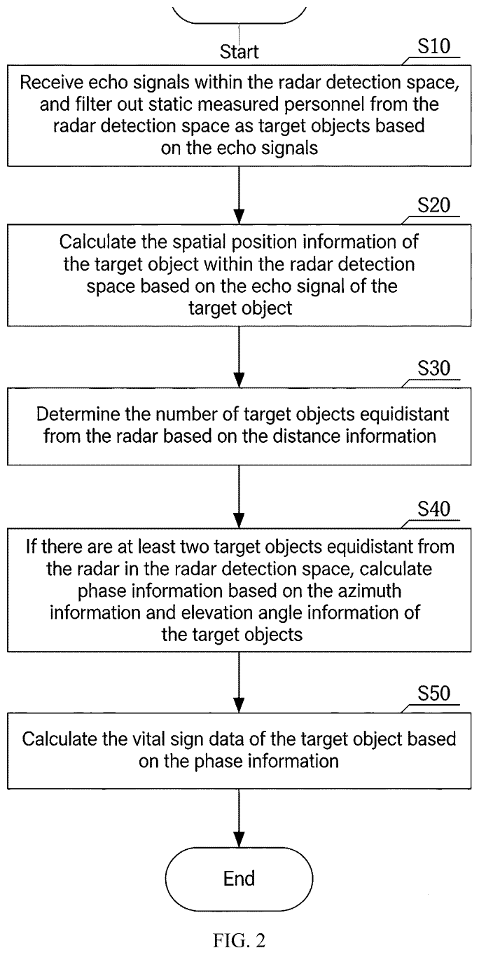

The antenna array layout of the millimeter wave radar of the present invention is shown in FIG. 2, where the virtual antenna array of the millimeter wave radar is a two-dimensional array composed of 12 antennas. Virtual antenna array is a technology that can achieve beamforming and beamforming by arranging antenna elements reasonably in space, in order to improve the efficiency of target positioning and signal reception.

Optionally, the millimeter wave radar of the present invention is based on top installation, usually installed on the ceiling or room ceiling, and only detects respiratory heart rate for stationary targets. In actual use, respiratory heart rate is only detected when a person is lying on a bed. If there is only one bed in the room, the radar should be installed on the ceiling directly above the bed as much as possible. If there are multiple beds in the room, the radar should be installed on the ceiling of the room in the middle position of the multiple beds. After the radar is installed and powered on, the installation height of the radar, the area where the bed is located, and the area where the door is located should be set according to the actual situation of the room, and it can be used normally.

Specifically, the memory 11 comprises at least one type of readable storage medium, including flash memory, hard disk, multimedia card, card type memory (such as SD or DX memory), magnetic memory, magnetic disk, optical disk, etc. Memory 11 may be an internal storage unit of server 1 in some embodiments, such as its hard disk. In other embodiments, the storage device 11 can also be an external storage device of the server 1, such as a plug-in hard drive, Smart Media Card (SMC), Secure Digital (SD) card, Flash Card, etc. equipped on the server 1.

Furthermore, the memory 11 may also include internal storage units of the server 1 as well as external storage devices. Memory 11 can not only be used to store application software and various data installed on server 1, such as the code of non-contact vital sign data monitoring program 10 based on millimeter wave radar, but also temporarily store data that has been or will be output.

In some embodiments, processor 12 may be a Central Processing Unit (CPU), controller, microcontroller, microprocessor, or other data processing chip used to run program code stored in memory 11 or process data, such as executing non-contact vital sign data monitoring program 10 based on millimeter wave radar.

Network interface 13 can optionally include standard wired interfaces and wireless interfaces (such as WI-FI interfaces), typically used to establish communication connections between system 1 and other electronic devices.

The network can be Internet, cloud network, Wi Fi network, personal network (PAN), local area network (LAN) and/or metropolitan area network (MAN). Various devices in the network environment can be configured to connect to the communication network according to various wired and wireless communication protocols. Examples of such wired and wireless communication protocols may include, but are not limited to, at least one of the following: transmission control protocol and Internet protocol (TCP/IP), user datagram protocol (UDP), hypertext transmission protocol (HTTP), file transfer protocol (FTP), ZigBee, EDGE, IEEE 802.11, optical fidelity (Li Fi), 802.16, IEEE 802.11s, IEEE 802.11g, multi hop communication, wireless access point (AP), device to device communication, cellular communication protocol and/or Bluetooth communication protocol or a combination thereof.

Optionally, the server may also include a user interface, which may include a display, an input unit such as a keyboard, and optional user interfaces may also include standard wired and wireless interfaces. Optionally, in some embodiments, the display may be an LED display, a liquid crystal display, a touch sensitive liquid crystal display, and an OLED (Organic Light Emitting Diode) touch sensor, among others. Among them, the display can also be referred to as a screen or display unit, used to display the information processed in server 1 and to display the visual user interface.

FIG. 1 only shows server 1 with components 11-13 and a non-contact vital sign data monitoring program 10 based on millimeter wave radar. Those skilled in the art can understand that the structure shown in FIG. 1 does not constitute a limitation on server 1, and may include fewer or more components than shown in the figure, or combine certain components, or arrange different components.

In this embodiment, processor 12 can be used to call the non-contact vital sign data monitoring program based on millimeter wave radar stored in memory 11 and perform the following operations:
Receive echo signals within the radar detection space and select static test subjects as target objects from the radar detection space based on the echo signals;
Calculate the spatial position information of the target object in the radar detection space based on its echo signal, which includes the distance information, azimuth information, and elevation information of the target unit compared to the radar;
Determine the number of target objects equidistant from the radar based on the spatial position information;
If there are at least two target objects equidistant from the radar in the detection space, calculate the phase information based on the azimuth and elevation information of the target objects;
Calculate the vital sign data of the target object based on the phase information, wherein the vital sign data includes at least one of respiratory rate and heart rate.

In one embodiment, processor 12 can be used to call the non-contact vital sign data monitoring program based on millimeter wave radar stored in memory 11 and perform the following operations:
Perform distance dimensional fast Fourier transform and Doppler dimensional fast Fourier transform on the echo signal to obtain distance Doppler data for each object in the thunderstorm detection space;
Perform incoherent accumulation on the range Doppler data of all virtual antennas of the radar to obtain the accumulated range Doppler data;
Perform two-dimensional constant false alarm detection on the accumulated distance Doppler data to extract objects with Doppler 0 from the radar detection space as candidate objects;
Select data with Doppler 0 from any virtual antenna and perform multi frame data accumulation processing to obtain slow Doppler dimensional distance Doppler data;
Perform static filtering on the distance Doppler data of the slow Doppler dimension to remove completely stationary background objects from the candidate objects, and use the remaining candidate objects as the target objects.

In one embodiment, processor 12 can be used to call the non-contact vital sign data monitoring program based on millimeter wave radar stored in memory 11 and perform the following operations:
Perform distance constant false alarm detection on the slow Doppler distance Doppler data of the target object, and calculate the distance unit of the target object compared to the radar;
Extract all antenna data of the target object from the distance Doppler data in the fast Doppler dimension;
Based on all antenna data of the target object, use the azimuth elevation joint angle measurement algorithm to calculate the azimuth and elevation angles of the target object.

In one embodiment, processor 12 can be used to call the non-contact vital sign data monitoring program based on millimeter wave radar stored in memory 11 and perform the following operations:
Extract phase information at the peak values of the azimuth frequency domain data and the elevation frequency domain data, respectively.

In one embodiment, processor 12 can be used to call the non-contact vital sign data monitoring program based on millimeter wave radar stored in memory 11 and perform the following operations:
Calculate phase information based on the distance information of the target object.

In one embodiment, processor 12 can be used to call the non-contact vital sign data monitoring program based on millimeter wave radar stored in memory 11 and perform the following operations:
Calculate phase information based on the distance unit data corresponding to Doppler 0 in the Doppler distance data.

In one embodiment, processor 12 can be used to call the non-contact vital sign data monitoring program based on millimeter wave radar stored in memory 11 and perform the following operations:
Accumulate multiple frames of data on the phase information to obtain phase change data of the target object;
Using a 6th order Butterworth filter designed based on the frequency range of respiratory rate to filter the phase change data of the target object and extract respiratory waveform data;
Perform fast Fourier transform on the respiratory waveform data to extract the respiratory cycle and calculate the respiratory frequency of the target object.

In one embodiment, processor 12 can be used to call the non-contact vital sign data monitoring program based on millimeter wave radar stored in memory 11 and perform the following operations:
Accumulate multiple frames of data on the phase information to obtain phase change data of the target object;
Using a 6th order Butterworth filter designed based on the frequency range of heart rate to filter the phase change data of the target object and extract heart rate waveform data;
Perform wavelet transform on the heart rate waveform data and then perform fast Fourier transform to extract the heart rate cycle, in order to calculate the heart rate of the target object.

Based on the hardware architecture of the non-contact vital sign data monitoring system based on millimeter wave radar mentioned above, an embodiment of the non-contact vital sign data monitoring method based on millimeter wave radar in the present invention is proposed. The non-contact vital sign data monitoring method based on millimeter wave radar of the present invention aims to improve the reliability of vital sign monitoring for multiple target objects.

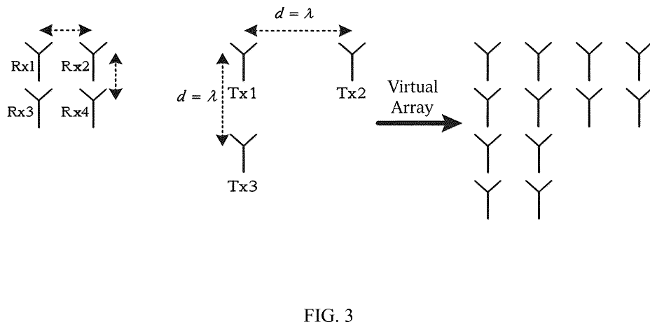

Referring to FIG. 3, FIG. 3 shows an embodiment of a non-contact vital sign data monitoring method based on millimeter wave radar according to the present invention. The non-contact vital sign data monitoring method based on millimeter wave radar includes the following steps:

[symbol] Receive echo signals within the radar detection space, and select static test subjects as target objects from the radar detection space based on the echo signals.

Specifically, millimeter wave radar systems first send millimeter wave signals, which interact with objects in the detection space. When these signals are reflected or scattered back by the target object, the radar receives the echo signal. Then, by analyzing these echo signals, the system can identify static personnel under test in the radar detection space and label them as target objects.

Among them, radar detection space refers to the three-dimensional spatial area used by millimeter wave radar systems for monitoring and detection. In the technical solution of this application, the radar detection space refers to the area covered and monitored by the millimeter wave radar system. Optionally, in different application scenarios, the radar detection space can be a closed room, an outdoor area, a traffic intersection, a hospital ward, and so on. Millimeter wave radar systems can be designed to accommodate detection spaces of different sizes and shapes to achieve various monitoring and detection tasks.

[symbol] Calculate the spatial position information of the target object in the radar detection space based on its echo signal, which includes the distance information, azimuth information, and elevation information of the target unit compared to the radar.

Specifically, once the target object is identified, the system will further analyze its echo signal to calculate the spatial position information of the target object within the radar detection space. This includes the distance, azimuth information, and elevation information of the target object relative to the radar. Among them, azimuth information and elevation information are parameters used to describe the position of the target object in the radar detection space. They are angle measurements in polar coordinate systems used to determine the direction and elevation angle of the target object relative to the radar. The azimuth information describes the horizontal direction of the target object relative to the radar position. The pitch angle information describes the vertical direction of the target object relative to the radar position.

Furthermore, spatial position information is typically represented using a Cartesian coordinate system, where X, Y, and Z represent the position of the target object in the horizontal, vertical, and distance directions, respectively. After obtaining the distance, azimuth, and elevation information of the target, the spatial position information of the target object can be obtained through triangulation or three-dimensional coordinate conversion based on the geometric parameters of the radar system and the measurement data of the target object.

[symbol] Determine the number of target objects equidistant from the radar based on the spatial position information.

Specifically, the system first distinguishes targets at different distances based on distance information. Then, the system further distinguishes these target objects based on their azimuth and elevation information. If there are at least two target objects at the same distance unit, and they do not overlap in azimuth and elevation angles, the system will consider these target objects equidistant from the radar and calculate their number as the number of target objects equidistant from the radar.

It can be understood that this method can effectively avoid aliasing or overlap between target objects and ensure that each target object can be accurately identified and monitored.

[symbol] If there are at least two target objects equidistant from the radar in the detection space, calculate the phase information based on the azimuth and elevation information of the target objects.

Specifically, if there are at least two target objects within the equidistant range of the radar, the system will calculate the phase information based on the azimuth and elevation information of the target objects. Phase information refers to the relative phase difference between millimeter wave signals reflected by different target objects.

It can be understood that when facing multiple equidistant target objects, azimuth and elevation information allow the target objects to be positioned in three-dimensional space. This means that even if multiple target objects have the same distance from the radar, their positions in the horizontal and vertical directions can be accurately distinguished. In this way, even if multiple target objects are equidistant from the radar, the radar system can still separate and recognize multiple target objects, effectively distinguishing and monitoring the respiratory rate and heart rate of multiple equidistant target objects, and improving the reliability of monitoring vital sign data for multiple target objects.

[symbol] Calculate the vital sign data of the target object based on the phase information, wherein the vital sign data includes at least one of respiratory rate and heart rate.

Specifically, when the human body breathes or the heart beats, the movement of the chest and heart can cause changes in the phase of millimeter wave signals. This change is usually manifested as a phase shift or rate of change in the signal. The phase change caused by breathing usually has different frequencies and characteristics from the phase change caused by heartbeat. Breathing typically has a lower frequency (usually between 0.1 Hz and 0.8 Hz), while heartbeat typically has a higher frequency (usually between 0.8 Hz and 2.5 Hz or higher). Based on the above principles, non-contact vital sign data monitoring can be achieved by monitoring the phase changes of echo signals caused by the breathing and heartbeat of the tested personnel, and calculating the respiratory frequency and/or heart rate of each tested personnel through methods such as fast Fourier transform.

It can be understood that the non-contact vital sign data monitoring method based on millimeter wave radar in the technical solution of the present application can not only achieve non-contact monitoring, but also effectively distinguish and monitor the respiratory frequency and/or heart rate of multiple equidistant target objects by introducing azimuth angle information and elevation angle information to obtain phase information when multiple target objects are detected in space, and then calculate the respiratory frequency and/or heart rate of each target object based on the obtained phase information to achieve non-contact vital sign data monitoring. Compared with traditional contact based vital sign monitoring methods or millimeter wave radar vital sign monitoring methods, the monitoring method of the present application can not only achieve non-contact monitoring, but also effectively distinguish and monitor the respiratory frequency and/or heart rate of multiple equidistant target objects, thereby improving the monitoring of vital sign data for multiple target objects. Reliability of monitoring. Even so, the monitoring method of the present technical solution has advantages such as non-contact monitoring and strong monitoring reliability.

In addition, the monitoring method based on the technical solution of the present application can simultaneously detect vital sign data of multiple people using only one device in one space, which helps to reduce the monitoring cost of multi object vital sign data monitoring and improve the cost-effectiveness of monitoring.

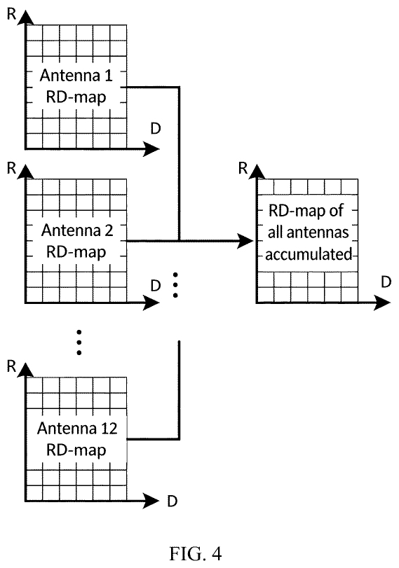

Please refer to FIGS. 4 to 6. In some embodiments, static personnel under test are selected as target objects from the radar detection space based on the echo signals, including the following steps:

[symbol] Perform Range FFT and Doppler FFT on the echo signal to obtain distance Doppler data for each object in the thunderstorm detection space.

Among them, in radar signal processing, the distance dimension is used to measure the distance between the target object and the radar, and the Doppler dimension is used to measure the velocity of the target object.

Specifically, in the technical solution of this application, the millimeter wave radar adopts the TD-MIMO signal transmission mode, with 3 transmitting antennas transmitting signals in a time-division multiplexing manner and 4 receiving antennas simultaneously receiving signals, forming 12 virtual antennas. Each antenna emits M chirps, each with N ADC sampling points. Chirp is a linear frequency modulation signal whose frequency varies linearly with time, which can improve the resolution and anti-interference ability of radar. ADC is an analog-to-digital converter that can convert analog signals into digital signals for subsequent signal processing. The ADC data volume of the current frame is 12*M*N.

In step S11, first perform N-point Range FFT on the ADC data to obtain distance information for each object. Among them, FFT is a transformation that can convert a signal into a frequency domain representation, used to extract the period or frequency of the signal. Range FFT is a method of measuring target distance by utilizing the relationship between the frequency variation of the chirp signal and the target distance.

Next, perform M-point Doppler FFT on the Range FFT to obtain the Range Doppler Dimensional Map (RD map), which has a data volume of N*M. These data can be used to represent the position and velocity information of each target object within the radar detection space. Among them, Doppler FFT is a method of measuring target velocity by utilizing the relationship between the frequency change of the echo signal caused by target motion and the target velocity.

[symbol] Perform incoherent accumulation on the distance Doppler data of all virtual antennas of the radar to obtain the accumulated distance Doppler data.

Specifically, please refer to FIG. 4. In the technical solution of this application, after obtaining the RD map of each antenna, the RD maps of 12 virtual antennas can be non coherently accumulated to obtain the accumulated RD map. Among them, incoherent accumulation is a method of adding the RD maps of different antennas or frames according to their amplitudes without considering phase differences.

It can be understood that non coherent accumulation can improve the signal-to-noise ratio of the target, making it easier to detect objects during constant false alarm detection in subsequent steps.

[symbol] Perform 2D Constant False Alarm Rate Detector (2D CFAR) on the accumulated distance Doppler data to extract objects with Doppler 0 from the radar detection space as candidate objects.

Among them, 2D CFAR detection is a method of constant false alarm detection in both distance and Doppler dimensions, which can determine the presence of a target based on the average value of each unit and surrounding units in the RD map.

Specifically, 2D CFAR detection is based on a given false alarm rate threshold, and applies a two-dimensional threshold judgment to the accumulated distance Doppler data, treating objects corresponding to data points that exceed the threshold as existing objects. Therefore, after completing 2D CFAR detection, objects in the radar detection space can be filtered out.

Furthermore, since respiratory and heart rate detection is only performed on stationary targets, objects with Doppler 0 are selected as candidate objects from the existing objects. Among them, a Doppler of 0 indicates that the target has no velocity change, that is, it is stationary.

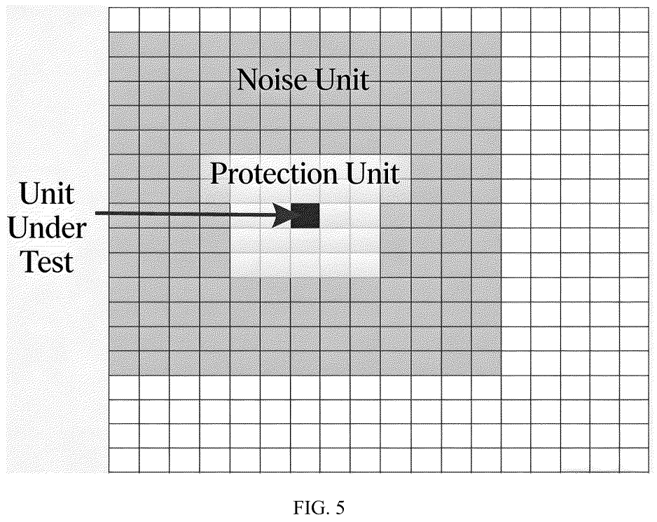

Please refer to FIG. 5, where the central unit represents the detected unit, which is the unit that needs to be determined whether it is the target or not; The intermediate unit represents the protection unit, which is adjacent to the detected unit and used to avoid edge effects of the target; The outer unit represents the noise unit, which is adjacent to the protection unit and used to estimate the average value of noise. When the detected unit is greater than a times the noise unit, the detection unit is detected, where a is a preset threshold parameter used to control the sensitivity of the detection.

[symbol] Select data with Doppler 0 from any virtual antenna and perform multi frame data accumulation processing to obtain slow Doppler dimensional distance Doppler data.

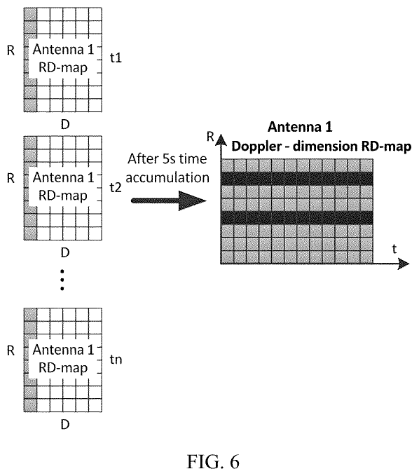

Specifically, please refer to FIG. 6. In this embodiment, the data with Doppler 0 of antenna 1 is selected and accumulated in multiple frames for 5 seconds to obtain the slow Doppler dimensional distance Doppler data (RD map). In this way, the temporal changes of these data can be obtained.

Among them, slow Doppler refers to the frequency variation of the echo signal caused by the small movement of the target, which is related to the target's breathing and heart rate.

It can be understood that by selecting data with Doppler 0 and accumulating multiple frames of data on them. By overlaying multiple frames of data, the observation time of the target object can be increased and the reliability of detection can be improved.

[symbol] Perform static filtering on the distance Doppler data of the slow Doppler dimension to remove completely stationary background objects from the candidate objects, and use the remaining candidate objects as the target objects.

Specifically, by performing static filtering on slow Doppler dimensional data (subtracting the average of multiple frames from each frame), the stability of the background signal can be utilized to remove completely stationary backgrounds, thereby retaining only the signal with changes, namely the target signal. The object corresponding to the target signal (i.e. the remaining candidate objects) can be considered as the target object.

Through the above steps S11 to S15, static personnel can be identified from the radar detection space. By combining distance, Doppler, and constant false alarm detection technologies, the accuracy and reliability of static targets can be improved.

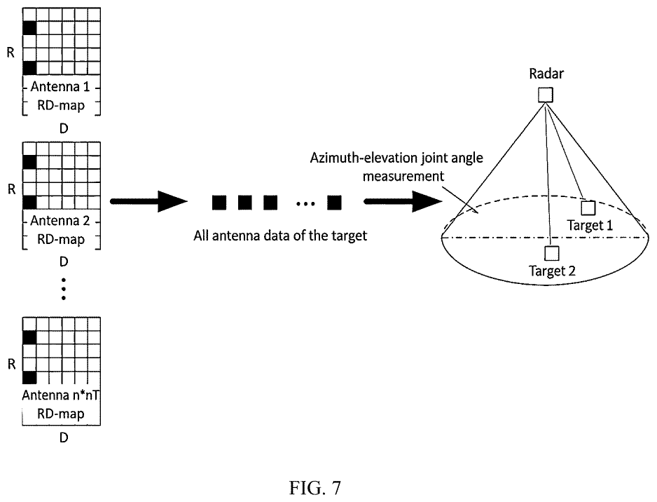

Please refer to FIG. 7. In some embodiments, calculating the spatial position information of the target object in the radar detection space based on its echo signal includes the following steps:

[symbol] Perform distance constant false alarm detection on the slow Doppler distance Doppler data of the target object, and calculate the distance unit of the target object compared to the radar.

Among them, distance based constant false alarm detection is a method of performing constant false alarm detection in the distance dimension, which can determine the presence of a target based on the average value of each unit and surrounding units in the slow Doppler spectrum.

Specifically, after performing constant false alarm detection on the slow Doppler distance Doppler data of the target object, the distance units of the stationary target (i.e. the target object) with breathing and heart rate can be extracted.

[symbol] Extract all antenna data of the target object from the distance Doppler data in the fast Doppler dimension.

Among them, the fast Doppler distance Doppler data is collected through a radar system, which includes the signal information received by various antennas. The signal received by each antenna includes the distance and Doppler frequency information of the target object, which is related to the characteristics of the target object in terms of distance and velocity.

Specifically, after determining the target object, echo signals related to the target object can be identified to confirm the data points associated with the target object. Then, it can be determined which antennas received this data. Next, all antenna data related to the target object can be extracted from the distance Doppler data in the fast Doppler dimension.

[symbol] Based on all antenna data of the target object, use the azimuth elevation joint angle measurement algorithm to calculate the azimuth and elevation angles of the target object.

Among them, the azimuth elevation joint angle measurement algorithm is a method used to calculate the azimuth and elevation angles of the target object in the radar detection space. The core principle of the azimuth elevation joint angle measurement algorithm is to use multiple antennas or sensors to measure the signals of the target object, and combine the phase and amplitude information of these signals to calculate the azimuth and elevation angles.

Specifically, based on all antenna data extracted from the target object, the azimuth elevation joint angle measurement algorithm is used to calculate the azimuth and elevation angles of the target object. Once the azimuth and elevation information is calculated, the three-dimensional position of the target object in the radar detection space can be obtained.

In some embodiments, azimuth information includes azimuth frequency domain data, and elevation information includes elevation frequency domain data.

Specifically, by performing an angular fast Fourier transform on the echo signal of the target object, the azimuth frequency domain data and elevation frequency domain data of the target object can be obtained. Among them, azimuth frequency domain data and elevation frequency domain data can display the amplitude and phase information of frequency components.

Correspondingly, extracting phase information based on the azimuth and elevation information of the target object includes extracting phase information at the peak of the azimuth frequency domain data and the elevation frequency domain data, respectively.

Among them, in frequency domain data, phase information is usually correlated with the peak value of frequency components. The peak represents the maximum strength of a signal at a specific frequency.

Specifically, for azimuth information, phase information can be extracted at the peak of the azimuth frequency domain data (the frequency component with the maximum amplitude). Similarly, for pitch angle information, phase information can be extracted at the peak of the pitch angle frequency domain data (the frequency component with the maximum amplitude).

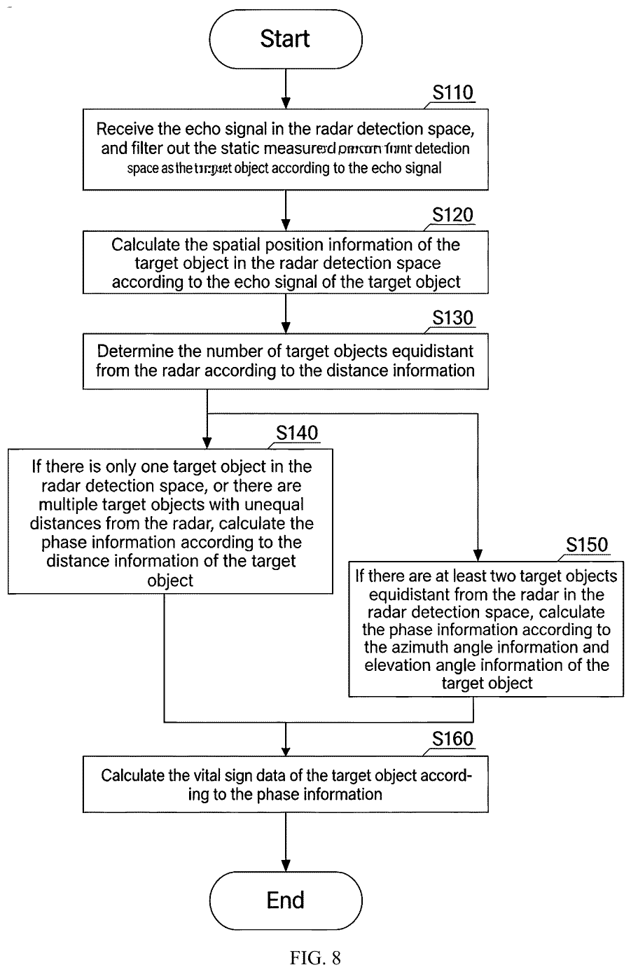

As shown in FIG. 8, in some embodiments, before calculating the vital sign data of the target object based on the phase information, if there is only one target object in the radar detection space, or if there are multiple target objects that are not equidistant from the radar, the method further comprises:
Calculate phase information based on the distance information of the target object.

Specifically, when there is only one target object or multiple target objects in the radar detection space, but the distances between the multiple target objects and the radar are not equal, the system will obtain their phase information based on the distance information of the target objects.

It can be understood that when there is only one object or multiple unevenly spaced target objects in the radar detection space, the radar can accurately identify multiple target objects based on distance information. Therefore, obtaining phase information based on distance information can reduce computational complexity, decrease system computational load, save computing resources, and improve computational efficiency while ensuring the reliability of monitoring vital signs of the target object.

In some embodiments, the distance information includes Doppler distance data.

Specifically, by performing Doppler fast Fourier transform on the echo signal of the target object, the Doppler distance data of the target object can be obtained. This Doppler distance data contains the distance information of the target object from the radar and the Doppler frequency information.

Furthermore, calculating phase information based on the distance information of the target object includes:
Calculate phase information based on the distance unit data corresponding to Doppler 0 in the Doppler distance data.

Among them, a Doppler of 0 corresponds to zero velocity of the target object relative to the radar, that is, the target object is stationary or has a very low velocity relative to the radar.

Specifically, by analyzing Doppler distance data, the distance unit data corresponding to Doppler 0 can be determined, which represents the position of all stationary or almost stationary target objects at the distance radar.

Furthermore, after determining the distance unit data corresponding to Doppler 0, phase information can be obtained by analyzing the phase components of Doppler distance data.

In some embodiments, calculating the respiratory rate of the target object based on the phase information comprises the following steps:

[symbol] Accumulate multiple frames of data on the phase information to obtain the phase change data of the target object.

Specifically, phase information from the target object can be accumulated in multiple frames, for example, by accumulating phase information for 10 seconds. By accumulating multiple frames, extract the phase change waveforms/curves (i.e. phase change curves) of each target object within multiple frame times.

Among them, the accumulation of multiple frames of data helps to increase the signal-to-noise ratio of the signal, thereby improving the accuracy of respiratory rate monitoring.

[symbol] Using a 6th order Butterworth filter designed based on the frequency range of respiratory rate, the phase change data of the target object is filtered to extract respiratory waveform data.

Among them, the Butterworth filter is a digital filter commonly used in signal processing, which enhances the signal within a specific frequency range while suppressing unwanted frequency components. The 6th order Butterworth filter designed through the frequency range of respiratory frequency can emphasize respiratory waveform signals and reduce noise.

Specifically, after filtering the phase change data, the obtained signal can be considered as respiratory waveform data, which reflects the periodic respiratory changes of the target object.

[symbol] Perform fast Fourier transform on the respiratory waveform data to extract the respiratory cycle and calculate the respiratory frequency of the target object.

Specifically, by performing Fast Fourier Transform (FFT) on the extracted respiratory waveform data, the frequency components of the target object's respiratory cycle can be extracted from the waveform. Based on the periodic changes in the frequency components of the respiratory cycle, the respiratory frequency of the target object can be calculated.

In addition, by analyzing the results of FFT, the amplitude and phase information corresponding to the respiratory frequency in the spectrum can also be extracted. Among them, the amplitude information of respiratory rate can be used to represent the intensity of breathing.

In some embodiments, calculating the heart rate of the target object based on the phase information comprises the following steps:

[symbol] Accumulate multiple frames of data on the phase information to obtain the phase change data of the target object.

Specifically, phase information from the target object can be accumulated in multiple frames, for example, by accumulating phase information for 10 seconds. By accumulating multiple frames, extract the phase change waveforms/curves (i.e. phase change curves) of each target object within multiple frame times.

Among them, the accumulation of multiple frames of data helps to increase the signal-to-noise ratio of the signal, thereby improving the accuracy of respiratory rate monitoring.

[symbol] Using a 6th order Butterworth filter designed based on the frequency range of heart rate to filter the phase change data of the target object and extract heart rate waveform data;

Among them, the Butterworth filter is a digital filter commonly used in signal processing, which enhances the signal within a specific frequency range while suppressing unwanted frequency components. The 6th order Butterworth filter designed through the frequency range of heart rate can emphasize the heartbeat waveform signal and reduce noise.

Specifically, after filtering the phase change data, the obtained signal can be considered as heart rate waveform data, which reflects the periodic changes in heart rate of the target object.

[symbol] Perform wavelet transform on the heart rate waveform data and then perform fast Fourier transform to extract the heart rate cycle, in order to calculate the heart rate of the target object.

Among them, wavelet transform is a transformation that can decompose signals into basis functions of different scales and frequencies, used to extract detailed features of signals or remove noise.

Specifically, by performing wavelet transform on the extracted respiratory waveform data and performing fast Fourier transform (FFT), the frequency components of the target object's heart rate cycle can be extracted from the waveform. Based on the periodic changes in the frequency components of the heart rate cycle, the heart rate of the target object can be calculated.

In addition, by analyzing the results of FFT, amplitude and phase information corresponding to the spectral center rate can also be extracted. Among them, the amplitude information of heart rate can be used to represent the intensity of heartbeat.

In some embodiments, if the number of target objects in the radar detection space exceeds a preset threshold, the radar monitoring system will not monitor the vital sign data of the target objects.

For example, the preset threshold can be set to 4.

It can be understood that when the number of target objects in a specific space (such as a ward) exceeds the set number of hospital beds (i.e. the set number), it indicates that there are more than expected personnel in the space. At this time, vital sign monitoring may detect vital sign data of non recorded personnel (patients), which may affect the security and reliability of the data. At this point, controlling the radar system to stop monitoring vital signs can not only save monitoring resources, but also ensure the purity of monitoring data.

In addition, the embodiments of the present invention also propose a computer-readable storage medium, which may be a hard disk, a multimedia card, an SD card, a flash memory card [symbol] Any one or any combination of read-only memory (ROM), erasable programmable read-only memory (EPROM), portable compact disc read-only memory (CD-ROM), USB memory, etc. The computer-readable storage medium includes a non-contact vital sign data monitoring program 10 based on millimeter wave radar. The specific implementation of the computer-readable storage medium of the present invention is roughly the same as the non-contact vital sign data monitoring method based on millimeter wave radar and the specific implementation of server 1 described above, and will not be repeated here.

Technicians in this field should understand that embodiments of the present invention can be provided as methods, systems, or computer program products. Therefore, the present invention can be implemented in the form of a fully hardware implementation, a fully software implementation, or a combination of software and hardware aspects. Moreover, the present invention may take the form of a computer program product implemented on one or more computer usable storage media (including but not limited to disk storage, CD-ROM, optical storage, etc.) containing computer usable program code.

The present invention is described with reference to flowcharts and/or block diagrams of methods, devices (systems), and computer program products according to embodiments of the present invention. It should be understood that each process and/or block in the flowchart and/or block diagram, as well as the combination of processes and/or blocks in the flowchart and/or block diagram, can be implemented by computer program instructions. These computer program instructions can be provided to processors of general-purpose computers, specialized computers, embedded processors, or other programmable data processing systems to generate a machine, such that the instructions executed by the processor of the computer or other programmable data processing system generate devices for implementing the functions specified in one or more processes in the flowchart and/or one or more boxes in the block diagram.

These computer program instructions can also be stored in computer-readable memory that can boot a computer or other programmable data processing system to work in a specific way, causing the instructions stored in the computer-readable memory to produce a manufactured product including instruction devices that implement the functions specified in a flowchart, one or more processes, and/or a block diagram, one or more boxes.

These computer program instructions can also be loaded onto a computer or other programmable data processing system, enabling a series of operational steps to be performed on the computer or other programmable system to generate computer implemented processing. The instructions executed on the computer or other programmable system provide steps for implementing the functions specified in a flowchart or multiple flowcharts and/or a block diagram or multiple boxes.

Although preferred embodiments of the present invention have been described, those skilled in the art may make additional changes and modifications to these embodiments once they have knowledge of the basic inventive concept. Therefore, the attached claims are intended to be interpreted as including preferred embodiments and all changes and modifications falling within the scope of the present invention.

Obviously, technicians in this field can make various modifications and variations to the present invention without departing from the spirit and scope of the invention. Thus, if these modifications and variations of the present invention fall within the scope of the claims and their equivalent technologies, the present invention is also intended to include these modifications and variations.

What is claimed is:

1. A non-contact vital sign data monitoring method based on millimeter wave radar, comprising:
receive echo signals within the radar detection space and select static test subjects as target objects from the radar detection space based on the echo signals;
calculate the spatial position information of the target object in the radar detection space based on its echo signal, which includes the distance information, azimuth information, and elevation information of the target unit compared to the radar;
determine the number of target objects equidistant from the radar based on the spatial position information;
if there are at least two target objects equidistant from the radar in the detection space, calculate the phase information based on the azimuth and elevation information of the target objects;
calculate the vital sign data of the target object based on the phase information, wherein the vital sign data includes at least one of respiratory rate and heart rate.

2. The non-contact vital sign data monitoring method based on millimeter wave radar according to claim 1, characterized in that the millimeter wave radar has multiple virtual antennas
selecting static personnel as target objects from the radar detection space based on the echo signals, including:
perform distance dimension fast Fourier transform and Doppler dimension fast Fourier transform on the echo signal to obtain distance Doppler data for each object in the radar detection space;
perform incoherent accumulation on the range Doppler data of all virtual antennas of the radar to obtain the accumulated range Doppler data;
perform two-dimensional constant false alarm detection on the accumulated distance Doppler data to extract objects with Doppler 0 from the radar detection space as candidate objects;
select data with Doppler 0 from any virtual antenna and perform multi frame data accumulation processing to obtain slow Doppler dimensional distance Doppler data;
perform static filtering on the distance Doppler data of the slow Doppler dimension to remove completely stationary background objects from the candidate objects, and use the remaining candidate objects as the target objects.

3. The non-contact vital sign data monitoring method based on millimeter wave radar according to claim 2, characterized in that the spatial position information of the target object in the radar detection space is calculated based on the echo signal of the target object, comprising:
perform distance constant false alarm detection on the slow Doppler distance Doppler data of the target object, and calculate the distance unit of the target object compared to the radar;
extract all antenna data of the target object from the distance Doppler data in the fast Doppler dimension;
based on all antenna data of the target object, use the azimuth elevation joint angle measurement algorithm to calculate the azimuth and elevation angles of the target object.

4. The non-contact vital sign data monitoring method based on millimeter wave radar according to claim 2, characterized in that the azimuth information includes azimuth frequency domain data, and the elevation information includes elevation frequency domain data;
extracting phase information based on the azimuth and elevation information of the target object, including:
extract phase information at the peak values of the azimuth frequency domain data and the elevation frequency domain data, respectively.

5. The non-contact vital sign data monitoring method based on millimeter wave radar according to claim 2, characterized in that, before calculating the vital sign data of the target object based on the phase information, if there is only one target object in the radar detection space, or if there are multiple target objects that are not equidistant from the radar, the method further comprises:
calculate phase information based on the distance information of the target object.

6. The non-contact vital sign data monitoring method based on millimeter wave radar according to claim 5, characterized in that the distance information includes Doppler distance data;
calculate phase information based on the distance information of the target object, including:
calculate phase information based on the distance unit data corresponding to Doppler 0 in the Doppler distance data.

7. The non-contact vital sign data monitoring method based on millimeter wave radar according to claim 1, characterized in that the respiratory frequency of the target object is calculated based on the phase information, comprising:
accumulate multiple frames of data on the phase information to obtain phase change data of the target object;
using a 6th order Butterworth filter designed based on the frequency range of respiratory rate to filter the phase change data of the target object and extract respiratory waveform data;
perform fast Fourier transform on the respiratory waveform data to extract the respiratory cycle and calculate the respiratory frequency of the target object.

8. The non-contact vital sign data monitoring method based on millimeter wave radar according to claim 1, characterized in that the heart rate of the target object is calculated based on the phase information, comprising:
Accumulate multiple frames of data on the phase information to obtain phase change data of the target object;
Using a 6th order Butterworth filter designed based on the frequency range of heart rate to filter the phase change data of the target object and extract heart rate waveform data;
Perform wavelet transform on the heart rate waveform data and then perform fast Fourier transform to extract the heart rate cycle, in order to calculate the heart rate of the target object.

9. A non-contact vital sign data monitoring system based on millimeter wave radar, characterized by comprising a millimeter wave radar, a memory, a processor, and a non-contact vital sign data monitoring program based on millimeter wave radar stored on the memory and executable on the processor,
when the processor executes the non-contact vital sign data monitoring program based on millimeter wave radar, it implements the non-contact vital sign data monitoring method based on millimeter wave radar as claimed in claim 1.

10. A computer-readable storage medium, characterized in that a non-contact vital sign data monitoring program based on millimeter wave radar is stored on the computer-readable storage medium; When the non-contact vital sign data monitoring program based on millimeter wave radar is executed by a processor, it implements the non-contact vital sign data monitoring method based on millimeter wave radar as claimed in claim 1.
